(19) United States
(12) Patent Application Publication
(10) Pub. No.: US 2025/0104323 A1
(43) Pub. Date: Mar. 27, 2025

(54) METHODS AND SYSTEMS FOR GENERATING THREE-DIMENSIONAL RENDERINGS OF A SCENE USING A MOBILE SENSOR ARRAY, SUCH AS NEURAL RADIANCE FIELD (NeRF) RENDERINGS

(71) Applicant: Proprio, Inc., Seattle, WA (US)

(72) Inventors: Thomas Ivan Nonn, Kenmore, WA (US); Adam Gabriel Jones, Seattle, WA (US); James Andrew Youngquist, Seattle, WA (US)

(21) Appl. No.: 18/892,323

(22) Filed: Sep. 20, 2024

Related U.S. Application Data
(60) Provisional application No. 63/584,691, filed on Sep. 22, 2023.

(51) Int. Cl. G06T 15/00; A61B 90/00; G06T 7/50; G06T 7/70; G06T 7/80

(52) U.S. Cl. CPC G06T 15/00; A61B 90/361; G06T 7/50; G06T 7/70; G06T 7/80; G06T 2207/10024; G06T 2207/30244

(57) ABSTRACT

Methods of generating three-dimensional (3D) views of a scene, such as a surgical scene, and associated systems and devices are disclosed herein. In some embodiments, a representative method includes moving a sensor array about a target volume and capturing RGB image data and depth data of the target volume with multiple cameras and a depth sensor of the sensor array, respectively. Poses of the RBG cameras and the depth sensor can be determined at each position. The captured RGB image data and the RGB camera poses can be used to train a radiance volume of a neural radiance field (NeRF) algorithm, and the depth data can be used to constrain the training of the NeRF algorithm. The NeRF algorithm can render a 3D image of the target volume based on a specified observer pose.

### CROSS-REFERENCE TO RELATED APPLICATION

This application claims the benefit of U.S. Provisional Patent Application No. 63/584,691, filed Sep. 22, 2023, and titled “METHODS AND SYSTEMS FOR GENERATING THREE-DIMENSIONAL RENDERINGS OF A SCENE USING A MOBILE SENSOR ARRAY, SUCH AS NEURAL RADIANCE FIELD (NeRF) RENDERINGS,” which is incorporated herein by reference in its entirety.

### TECHNICAL FIELD

The present technology generally relates to methods and systems for generating three-dimensional (3D) views of a scene using a mobile sensor array and, more particularly, to generating neural radiance field (NeRF) renderings and/or Gaussian splatting renderings of a surgical scene, such as a spinal surgical scene.

### BACKGROUND

In a mediated-reality system, an image processing system adds, subtracts, and/or modifies visual information representing an environment. For surgical applications, a mediated-reality system may enable a surgeon to view a surgical site from a desired perspective together with contextual information that assists the surgeon in more efficiently and precisely performing surgical tasks. When performing surgeries, surgeons often rely on previously-captured or initial three-dimensional images of the patient's anatomy, such as computed tomography (CT) scan images and/or magnetic resonance imaging (MRI) scan images. However, the usefulness of such initial images is limited because the images cannot be easily integrated into the operative procedure. For example, because the images are captured in an initial session, the relative anatomical positions captured in the initial images may vary from their actual positions during the operative procedure. Furthermore, to make use of the initial images during the surgery, the surgeon must divide their attention between the surgical field and a display of the initial images. Navigating between different layers of the initial images may also require significant attention that takes away from the surgeon's focus on the operation.

High-performance display technology and graphics hardware enable immersive three-dimensional environments to be presented in real-time with high levels of detail. Currently these immersive environments are primarily limited to the context of video games and simulations where the environment is rendered from a game engine with assets and textures created by artists during development. However, these environments fall short of photorealistic appearance and this virtual world paradigm does not allow for mediated interactions with the real world around the user. In applications where users interact with their physical environment, streaming of video data (often from a single imager) is used. However, the perspective and motion of the user is tied directly to that of the physical imager. Furthermore, merely overlaying information on the video stream lacks the immersion and engagement provided by a synthesized viewpoint that accurately recreates the real world, while seamlessly integrating additional information sources.

Some systems synthesize input images from a set of imagers to generate a view of a scene using deep learning methods to generate photorealistic results. Such deep learning methods include neural radiance field rendering (NeRF), Gaussian splatting (e.g., three-dimensional (3D) Gaussian splatting), neural lumigraph rendering (NLR), and bundle-adjusting neural radiance field rendering (BaRF). However, these methods are encumbered by the computational intensity of model training, which can take many hours to days on a single graphics processing unit (GPU). Furthermore, these methods do not support real-time rendering of the neural scene representation. Additionally, such methods can require tens to hundreds of input views and therefore require large arrays of cameras to capture images.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic view of an imaging system in accordance with embodiments of the present technology.

FIG. 2 is a perspective view of an environment employing the imaging system of FIG. 1 in accordance with embodiments of the present technology.

FIG. 3 is an isometric view of a portion of the imaging system of FIG. 1 illustrating four cameras of a sensor array of the imaging system in accordance with embodiments of the present technology.

FIG. 4 is a flow diagram of a process or method for generating a three-dimensional (3D) view of a scene from data captured by the sensor array of the imaging system of FIGS. 1-3 in accordance with embodiments of the present technology.

FIG. 5 is a partially-schematic perspective view of the imaging system of FIGS. 1-3 in accordance with embodiments of the present technology.

FIG. 6 is a partially-schematic perspective view of the imaging system of FIGS. 1-3 with the sensor array moved via a robotic mover and positioned at multiple different positions relative to the scene in accordance with embodiments of the present technology.

FIGS. 7A-7C are schematic perspective views of different 3D depth maps captured by sensor array at different positions about the scene in accordance with embodiments of the present technology.

FIG. 7D is a schematic perspective view of a combined or unified depth map that combines the different depth maps shown in FIGS. 7A-7C in accordance with embodiments of the present technology.

FIG. 8A illustrates several rendered images of the scene in accordance with embodiments of the present technology.

FIG. 8B illustrates a finally rendered output image of the scene in accordance with embodiments of the present technology.

FIGS. 9A-9D are different views of a user interface visible to a user of the imaging system of FIGS. 1-3 via a display device in accordance with embodiments of the present technology.

### DETAILED DESCRIPTION

Aspects of the present technology are directed generally to methods of generating three-dimensional (3D) views of a scene, such as a surgical scene, with a mobile sensor array and associated systems and devices. In some embodiments, a representative method includes moving a sensor array about a target volume via a robotically-controlled mover (e.g., a robotic arm) to/through multiple positions, and capturing RGB image data and depth data of the target volume with multiple cameras and a depth sensor of the sensor array, respectively, at each of the multiple positions. The method can include determining poses of the RBG cameras and poses of the depth sensor at the individual positions. The captured RGB image data can be inserted as training data into a radiance volume of a neural radiance field (NeRF) algorithm based on the determined poses of the RGB cameras. The depth data can be combined into a high-fidelity unified depth map (e.g., a point cloud) based on the determined poses of the depth sensor. The NeRF algorithm can then train the radiance volume based on the captured RGB data while constraining the training based on the unified depth map, and render a 3D image of the target volume based on a specified observer pose. The observer pose can be a novel perspective of the target volume that, for example, does not correspond to a physical position of any of the RGB cameras. Additionally, the rendered 3D image can be a photorealistic image. In some embodiments, in addition to or alternatively to a NeRF algorithm, the representative method can utilize a Gaussian splatting (e.g., three-dimensional (3D) Gaussian splatting) algorithm.

In some aspects of the present technology, registration transformations are initially determined between reference frames of the RGB cameras, a reference frame of the depth sensor, a reference frame of the sensor array, a reference frame of the robotically-controlled mover, and a reference frame of the target volume. The reference frames can be used to quickly and accurately determine the poses of the of the RBG cameras and the poses of the depth sensor. Accordingly, the system can maintain homography even as the robotically-controlled mover moves the sensor array about the scene. This can significantly increase the processing speed of the rendering as compared to conventional systems, providing for real-time or near real-time rendering of the 3D image of the target volume.

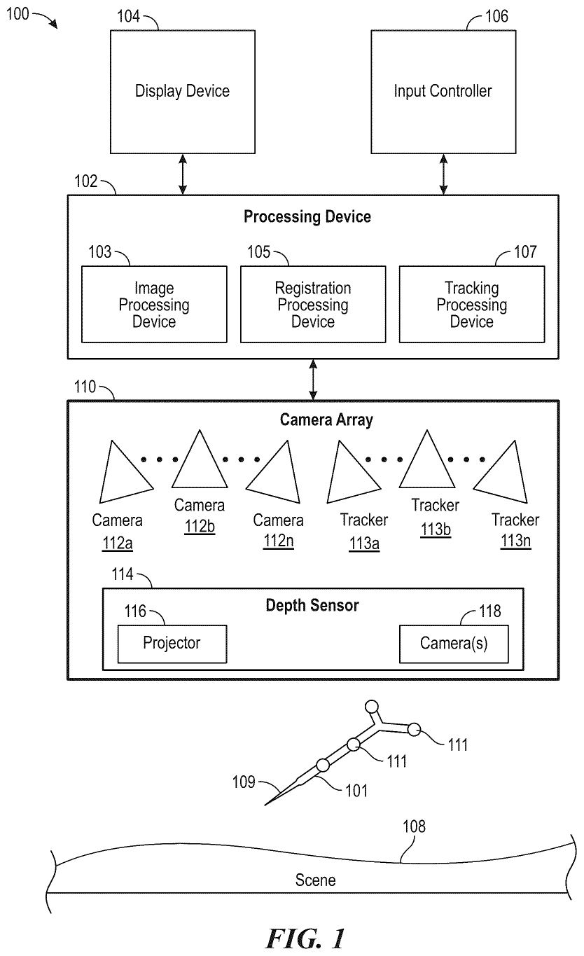

Specific details of several embodiments of the present technology are described herein with reference to FIGS. 1-9D. The present technology, however, can be practiced without some of these specific details. In some instances, well-known structures and techniques often associated with sensor arrays, RGB imaging, depth sensing, neural radiance field (NeRF) algorithms, Gaussian splatting algorithms, and processing, registration processes, and the like have not been shown in detail so as not to obscure the present technology.

The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section. Moreover, although frequently described in the context of imaging and rendering a surgical scene, and more particularly a spinal surgical scene, the present technology can be used to image, render, etc., other types of scenes.

The accompanying Figures depict embodiments of the present technology and are not intended to be limiting of its scope. Depicted elements are not necessarily drawn to scale, and various elements can be arbitrarily enlarged to improve legibility. Component details can be abstracted in the figures to exclude details as such details are unnecessary for a complete understanding of how to make and use the present technology. Many of the details, dimensions, angles, and other features shown in the Figures are merely illustrative of particular embodiments of the disclosure. Accordingly, other embodiments can have other dimensions, angles, and features without departing from the spirit or scope of the present technology.

The headings provided herein are for convenience only and should not be construed as limiting the subject matter disclosed. To the extent any materials incorporated herein by reference conflict with the present disclosure, the present disclosure controls.

### I. SELECTED EMBODIMENTS OF IMAGING SYSTEMS

FIG. 1 is a schematic view of an imaging system 100 (“system 100”) in accordance with embodiments of the present technology. In some embodiments, the system 100 can be a synthetic augmented reality system, a virtual-reality imaging system, an augmented-reality imaging system, a mediated-reality imaging system, and/or a non-immersive computational imaging system. In the illustrated embodiment, the system 100 includes a processing device 102 that is communicatively coupled to one or more display devices 104, one or more input controllers 106, and a sensor array 110 (e.g., a camera array, a sensor head, and/or the like). In other embodiments, the system 100 can comprise additional, fewer, or different components. In some embodiments, the system 100 includes some features that are generally similar or identical to those of the mediated-reality imaging systems disclosed in (i) U.S. patent application Ser. No. 16/586,375, filed Sep. 27, 2019, titled “CAMERA ARRAY FOR A MEDIATED-REALITY SYSTEM,” and/or (ii) U.S. patent application Ser. No. 15/930,305, filed May 12, 2020, and titled “METHODS AND SYSTEMS FOR IMAGING A SCENE, SUCH AS A MEDICAL SCENE, AND TRACKING OBJECTS WITHIN THE SCENE,” each of which is incorporated herein by reference in its entirety.

In the illustrated embodiment, the sensor array 110 includes a plurality of cameras 112 (identified individually as cameras 112a-112n; which can also be referred to as first cameras) that can each capture images of a scene 108 (e.g., first image data) from a different perspective. The scene 108 can include for example, a patient undergoing surgery (e.g., spinal surgery) and/or another medical procedure. In other embodiments, the scene 108 can be another type of scene. The sensor array 110 can further include dedicated object tracking hardware 113 (e.g., including individually identified trackers 113a-113n) that captures positional data of one more objects, such as an instrument 101 (e.g., a surgical instrument or tool) having a tip 109, to track the movement and/or orientation of the objects through/in the scene 108. In some embodiments, the cameras 112 and the trackers 113 are positioned at fixed locations and orientations (e.g., poses) relative to one another. For example, the cameras 112 and the trackers 113 can be structurally secured by/to a mounting structure (e.g., a common frame) at predefined fixed locations and orientations. In some embodiments, the cameras 112 are positioned such that neighboring cameras 112 share overlapping views of the scene 108. In general, the position of the cameras 112 can be selected to maximize clear and accurate capture of all or a selected portion of the scene 108. Likewise, the trackers 113 can be positioned such that neighboring trackers 113 share overlapping views of the scene 108. Therefore, all or a subset of the cameras 112 and the trackers 113 can have different extrinsic parameters, such as position and orientation (e.g., pose).

In some embodiments, the cameras 112 in the sensor array 110 are synchronized to capture images of the scene 108 simultaneously (within a threshold temporal error). In some embodiments, all or a subset of the cameras 112 are light field, plenoptic, and/or RGB cameras that capture information about the light field emanating from the scene 108 (e.g., information about the intensity of light rays in the scene 108 and also information about a direction the light rays are traveling through space). In some embodiments, image data from the cameras 112 can be used to reconstruct a light field of the scene 108. More specifically, the cameras 112 can be RGB cameras that capture a combined image data set for reconstructing a light field of the scene 108. Therefore, in some embodiments the images captured by the cameras 112 encode depth information representing a surface geometry of the scene 108. In some embodiments, the cameras 112 are substantially identical. In other embodiments, the cameras 112 include multiple cameras of different types. For example, different subsets of the cameras 112 can have different intrinsic parameters such as focal length, sensor type, optical components, and the like. The cameras 112 can have charge-coupled device (CCD) and/or complementary metal-oxide semiconductor (CMOS) image sensors and associated optics. Such optics can include a variety of configurations including lensed or bare individual image sensors in combination with larger macro lenses, micro-lens arrays, prisms, and/or negative lenses. For example, the cameras 112 can be separate light field cameras each having their own image sensors and optics. In other embodiments, some or all of the cameras 112 can comprise separate microlenslets (e.g., lenslets, lenses, microlenses) of a microlens array (MLA) that share a common image sensor. In other embodiments, some or all of the cameras 112 can be RGB (e.g., color) cameras having visible imaging sensors that together provide a light field data set of the scene 108.

In some embodiments, the trackers 113 are imaging devices, such as infrared (IR) cameras that can capture images of the scene 108 from a different perspective compared to other ones of the trackers 113. Accordingly, the trackers 113 and the cameras 112 can have different spectral sensitives (e.g., infrared vs. visible wavelength). In some embodiments, the trackers 113 capture image data of a plurality of optical markers (e.g., fiducial markers, marker balls) in the scene 108, such as markers 111 coupled to the instrument 101.

In the illustrated embodiment, the sensor array 110 further includes a depth sensor 114. In some embodiments, the depth sensor 114 includes (i) one or more projectors 116 that project a structured light pattern onto/into the scene 108 and (ii) one or more depth cameras 118 (which can also be referred to as second cameras) that capture second image data of the scene 108 including the structured light projected onto the scene 108 by the projector 116. The projector 116 can project a speckled pattern or a pattern of dots, for example. The projector 116 and the depth cameras 118 can operate in the same wavelength and, in some embodiments, can operate in a wavelength different than the cameras 112. For example, the cameras 112 can capture the first image data in the visible spectrum, while the depth cameras 118 capture the second image data in the infrared spectrum. In some embodiments, the depth cameras 118 have a resolution that is less than a resolution of the cameras 112. For example, the depth cameras 118 can have a resolution that is less than 70%, 60%, 50%, 40%, 30%, or 20% of the resolution of the cameras 112. In other embodiments, the depth sensor 114 can include other types of dedicated depth detection hardware (e.g., a LiDAR detector) for determining the surface geometry of the scene 108. In other embodiments, the sensor array 110 can omit the projector 116 and/or the depth cameras 118.

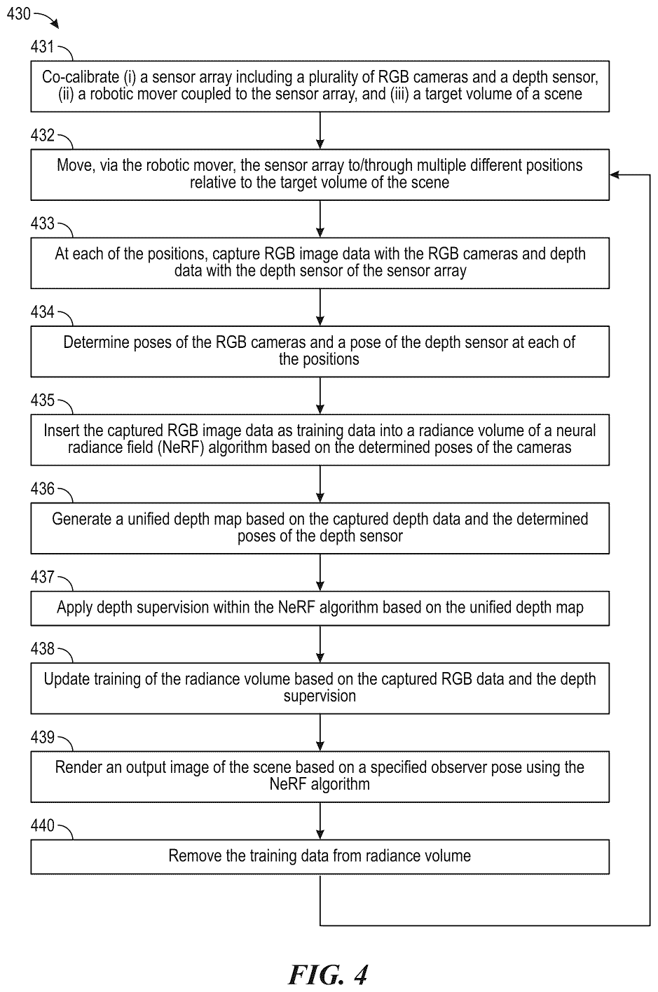

In the illustrated embodiment, the processing device 102 includes an image processing device 103 (e.g., an image processor, an image processing module, an image processing unit), a registration processing device 105 (e.g., a registration processor, a registration processing module, a registration processing unit), and a tracking processing device 107 (e.g., a tracking processor, a tracking processing module, a tracking processing unit). The image processing device 103 can (i) receive the first image data captured by the cameras 112 (e.g., light field images, light field image data, RGB images) and depth information from the depth sensor 114 (e.g., the second image data captured by the depth cameras 118), and (ii) process the image data and depth information to synthesize (e.g., generate, reconstruct, render) a three-dimensional (3D) output image of the scene 108 corresponding to a virtual camera perspective (e.g., a novel camera perspective). The output image can correspond to an approximation of an image of the scene 108 that would be captured by a camera placed at an arbitrary position and orientation corresponding to the virtual camera perspective. In some embodiments, the image processing device 103 can further receive and/or store calibration data for the cameras 112 and/or the depth cameras 118 and synthesize the output image based on the image data, the depth information, and/or the calibration data. More specifically, the depth information and the calibration data can be used/combined with the images from the cameras 112 to synthesize the output image as a 3D (or stereoscopic 2D) rendering of the scene 108 as viewed from the virtual camera perspective. In some embodiments, the image processing device 103 can synthesize the output image using any of the methods disclosed in U.S. patent application Ser. No. 16/457,780, filed Jun. 28, 2019, and titled “SYNTHESIZING AN IMAGE FROM A VIRTUAL PERSPECTIVE USING PIXELS FROM A PHYSICAL IMAGER ARRAY WEIGHTED BASED ON DEPTH ERROR SENSITIVITY,” which is incorporated herein by reference in its entirety. In other embodiments, the image processing device 103 can generate the virtual camera perspective based only on the images captured by the cameras 112 without utilizing depth information from the depth sensor 114. For example, the image processing device 103 can generate the virtual camera perspective by interpolating between the different images captured by one or more of the cameras 112. In some embodiments described in further detail below with reference to FIGS. 4-9D, the image processing device 103 can utilize a neural radiance field (NeRF) rendering algorithm to synthesize and render an output image of the scene 108 based on RGB images captured by the cameras 112 and depth data captured by the depth sensor 114.

The image processing device 103 can synthesize the output image from images captured by a subset (e.g., two or more) of the cameras 112 in the sensor array 110, and does not necessarily utilize images from all of the cameras 112. For example, for a given virtual camera perspective, the processing device 102 can select a stereoscopic pair of images from two of the cameras 112. In some embodiments, such a stereoscopic pair can be selected to be positioned and oriented to most closely match the virtual camera perspective. In some embodiments, the image processing device 103 (and/or the depth sensor 114) estimates a depth for each surface point of the scene 108 relative to a common origin to generate a point cloud and/or a 3D mesh that represents the surface geometry of the scene 108. Such a representation of the surface geometry can be referred to as a surface reconstruction, a 3D reconstruction, a 3D surface reconstruction, a depth map, a depth surface, and/or the like. In some embodiments, the depth cameras 118 of the depth sensor 114 detect the structured light projected onto the scene 108 by the projector 116 to estimate depth information of the scene 108. In some embodiments, the image processing device 103 estimates depth from multiview image data from the cameras 112 using techniques such as light field correspondence, stereo block matching, photometric symmetry, correspondence, defocus, block matching, texture-assisted block matching, structured light, and the like, with or without utilizing information collected by the depth sensor 114. In other embodiments, depth may be acquired by a specialized set of the cameras 112 performing the aforementioned methods in another wavelength.

In some embodiments, the registration processing device 105 receives and/or stores initial image data, such as image data of a three-dimensional volume of a patient (3D image data). The image data can include, for example, computerized tomography (CT) scan data, magnetic resonance imaging (MRI) scan data, ultrasound images, fluoroscope images, and/or other medical or other image data. The image data can be segmented or unsegmented. The registration processing device 105 can register the initial image data to the real-time images captured by the cameras 112 and/or the depth sensor 114 by, for example, determining one or more transforms/transformations/mappings between the two. The processing device 102 (e.g., the image processing device 103) can then apply the one or more transformations to the initial image data such that the initial image data can be aligned with (e.g., overlaid on) the output image of the scene 108 in real-time or near real time on a frame-by-frame basis, even as the virtual perspective changes. That is, the image processing device 103 can fuse the initial image data with the real-time output image of the scene 108 to present a mediated-reality view that enables, for example, a surgeon to simultaneously view a surgical site in the scene 108 and the underlying 3D anatomy of a patient undergoing an operation. In some embodiments, the registration processing device 105 can register the initial image data to the real-time images by using any of the methods disclosed in U.S. patent application Ser. No. 17/140,885, filed Jan. 4, 2021, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” which is incorporated by reference herein in its entirety.

In some embodiments, the tracking processing device 107 processes positional data captured by the trackers 113 to track objects (e.g., the instrument 101) within the vicinity of the scene 108. For example, the tracking processing device 107 can determine the position of the markers 111 in the 2D images captured by two or more of the trackers 113, and can compute the 3D position of the markers 111 via triangulation of the 2D positional data. More specifically, in some embodiments the trackers 113 include dedicated processing hardware for determining positional data from captured images, such as a centroid of the markers 111 in the captured images. The trackers 113 can then transmit the positional data to the tracking processing device 107 for determining the 3D position of the markers 111. In other embodiments, the tracking processing device 107 can receive the raw image data from the trackers 113. In a surgical application, for example, the tracked object can comprise a surgical instrument, an implant, a hand or arm of a physician or assistant, and/or another object having the markers 111 mounted thereto. In some embodiments, the processing device 102 can recognize the tracked object as being separate from the scene 108, and can apply a visual effect to the 3D output image to distinguish the tracked object by, for example, highlighting the object, labeling the object, and/or applying a transparency to the object.

In some embodiments, functions attributed to the processing device 102, the image processing device 103, the registration processing device 105, and/or the tracking processing device 107 can be practically implemented by two or more physical devices. For example, in some embodiments a synchronization controller (not shown) controls images displayed by the projector 116 and sends synchronization signals to the cameras 112 to ensure synchronization between the cameras 112 and the projector 116 to enable fast, multi-frame, multicamera structured light scans. Additionally, such a synchronization controller can operate as a parameter server that stores hardware specific configurations such as parameters of the structured light scan, camera settings, and camera calibration data specific to the camera configuration of the sensor array 110. The synchronization controller can be implemented in a separate physical device from a display controller that controls the display device 104, or the devices can be integrated together.

The processing device 102 can comprise a processor and a non-transitory computer-readable storage medium that stores instructions that when executed by the processor, carry out the functions attributed to the processing device 102 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The virtual camera perspective is controlled by an input controller 106 that can update the virtual camera perspective based on user driven changes to the camera's position and rotation. The output images corresponding to the virtual camera perspective can be outputted to the display device 104. In some embodiments, the image processing device 103 can vary the perspective, the depth of field (e.g., aperture), the focus plane, and/or another parameter of the virtual camera (e.g., based on an input from the input controller) to generate different 3D output images without physically moving the sensor array 110. The display device 104 can receive output images (e.g., the synthesized 3D rendering of the scene 108) and display the output images for viewing by one or more viewers. In some embodiments, the processing device 102 receives and processes inputs from the input controller 106 and processes the captured images from the sensor array 110 to generate output images corresponding to the virtual perspective in substantially real-time or near real-time as perceived by a viewer of the display device 104 (e.g., at least as fast as the frame rate of the sensor array 110).

Additionally, the display device 104 can display a graphical representation on/in the image of the virtual perspective of any (i) tracked objects within the scene 108 (e.g., a surgical instrument) and/or (ii) registered or unregistered initial image data. That is, for example, the system 100 (e.g., via the display device 104) can blend augmented data into the scene 108 by overlaying and aligning information on top of “passthrough” images of the scene 108 captured by the cameras 112 and/or generated by images captured by the cameras 112. Moreover, the system 100 can create a mediated-reality experience where the scene 108 is reconstructed using light field image data of the scene 108 captured by the cameras 112, and where instruments are virtually represented in the reconstructed scene via information from the trackers 113. Additionally or alternatively, the system 100 can remove the original scene 108 and completely replace it with a registered and representative arrangement of the initial image data, thereby removing information in the scene 108 that is not pertinent to a user's task.

The display device 104 can comprise, for example, a head-mounted display device, a monitor, a computer display, and/or another display device. In some embodiments, the input controller 106 and the display device 104 are integrated into a head-mounted display device and the input controller 106 comprises a motion sensor that detects position and orientation of the head-mounted display device. In some embodiments, the system 100 can further include a separate tracking system (not shown), such an optical tracking system, for tracking the display device 104, the instrument 101, and/or other components within the scene 108. Such a tracking system can detect a position of the head-mounted display device 104 and input the position to the input controller 106. The virtual camera perspective can then be derived to correspond to the position and orientation of the head-mounted display device 104 in the same reference frame and at the calculated depth (e.g., as calculated by the depth sensor 114) such that the virtual perspective corresponds to a perspective that would be seen by a viewer wearing the head-mounted display device 104. Thus, in such embodiments the head-mounted display device 104 can provide a real-time rendering of the scene 108 as it would be seen by an observer without the head-mounted display device 104. Alternatively, the input controller 106 can comprise a user-controlled control device (e.g., a mouse, pointing device, handheld controller, gesture recognition controller) that enables a viewer to manually control the virtual perspective displayed by the display device 104.

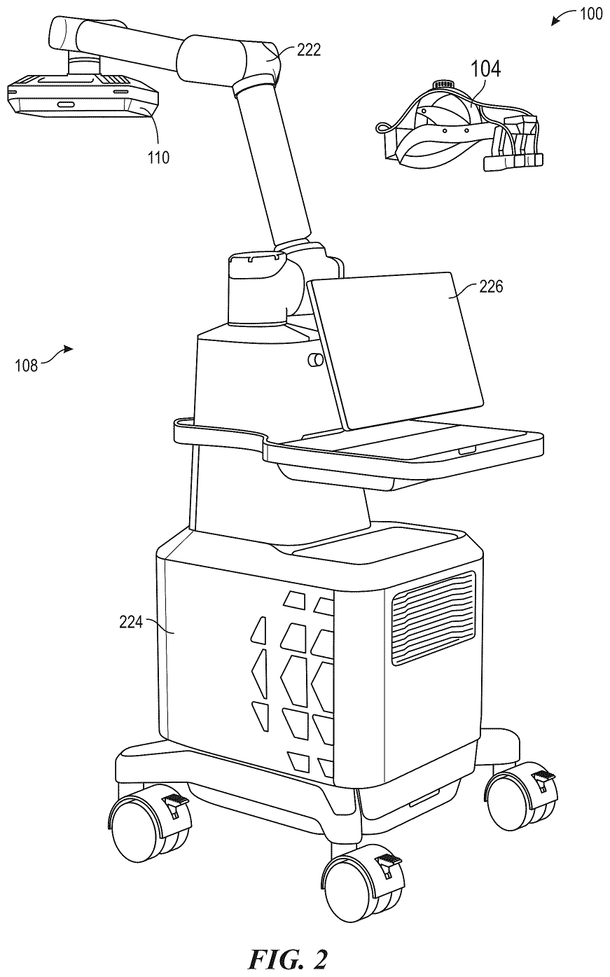

FIG. 2 is a perspective view of an environment (e.g., a surgical environment) employing the system 100 (e.g., for a surgical application) in accordance with embodiments of the present technology. In the illustrated embodiment, the sensor array 110 is positioned over the scene 108 (e.g., a surgical site) and supported/positioned via a mover 222 that is operably coupled to a workstation 224. In some embodiments, the mover 222 is manually movable to position the sensor array 110 while, in other embodiments, the mover 222 is robotically controlled in response to the input controller 106 (FIG. 1) and/or another controller. Accordingly, the mover 222 can be referred to as a robotic mover, a robotic arm, a robotically-controlled arm, and/or the like. The mover 222 allows the sensor array 110 to be precisely moved relative to the scene 108 such that the sensor array 110 is mobile relative to the scene 108.

In the illustrated embodiment, the display device 104 is a head-mounted display device (e.g., a virtual reality headset, augmented reality headset). The workstation 224 can include a computer to control various functions of the processing device 102, the display device 104, the input controller 106, the sensor array 110, and/or other components of the system 100 shown in FIG. 1. Accordingly, in some embodiments the processing device 102 and the input controller 106 are each integrated in the workstation 224. In some embodiments, the workstation 224 includes a secondary display 226 that can display a user interface for performing various configuration functions, a mirrored image of the display on the display device 104, and/or other useful visual images/indications. In other embodiments, the system 100 can include more or fewer display devices. For example, in addition to (or alternatively to) the display device 104 and the secondary display 226, the system 100 can include another display (e.g., a medical grade computer monitor) visible to the user wearing the display device 104.

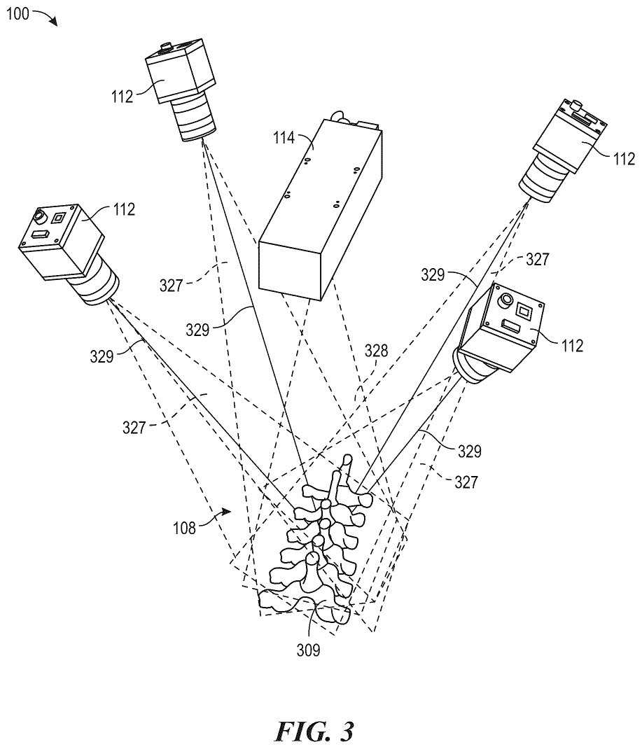

FIG. 3 is an isometric view of a portion of the system 100 illustrating four of the cameras 112 in accordance with embodiments of the present technology. Other components of the system 100 (e.g., other portions of the sensor array 110, the processing device 102, etc.) are not shown in FIG. 3 for the sake of clarity. In the illustrated embodiment, each of the cameras 112 has a field of view 327 and a focal axis 329. Likewise, the depth sensor 114 can have a field of view 328 aligned with a portion of the scene 108. The cameras 112 can be oriented such that the fields of view 327 are aligned with a portion of the scene 108 and at least partially overlap one another to together define an imaging volume. In some embodiments, some or all of the field of views 327, 328 at least partially overlap. For example, in the illustrated embodiment the fields of view 327, 328 converge toward a common measurement volume including a portion of a spine 309 of a patient (e.g., a human patient) located in/at the scene 108. In some embodiments, the cameras 112 are further oriented such that the focal axes 329 converge to a common point in the scene 108. In some aspects of the present technology, the convergence/alignment of the focal axes 329 can generally maximize disparity measurements between the cameras 112. In some embodiments, the cameras 112 and the depth sensor 114 are fixedly positioned relative to one another (e.g., rigidly mounted to a common frame) such that a relative positioning of the cameras 112 and the depth sensor 114 relative to one another is known and/or can be readily determined via a calibration process. In other embodiments, the system 100 can include a different number of the cameras 112 and/or the cameras 112 can be positioned differently relative to another.

Referring to FIGS. 1-3 together, in some aspects of the present technology the system 100 can generate a digitized view of the scene 108 that provides a user (e.g., a surgeon) with increased “volumetric intelligence” of the scene 108. For example, the digitized scene 108 can be presented to the user from the perspective, orientation, and/or viewpoint of their eyes such that they effectively view the scene 108 as though they were not viewing the digitized image (e.g., as though they were not wearing the head-mounted display 104). However, the digitized scene 108 permits the user to digitally rotate, zoom, crop, or otherwise enhance their view to, for example, facilitate a surgical workflow. Likewise, initial image data, such as CT scans and/or MRI data, can be registered to and overlaid over the image of the scene 108 to allow a surgeon to view these data sets together. Such a fused view can allow the surgeon to visualize aspects of a surgical site that may be obscured in the physical scene 108—such as regions of bone and/or tissue that have not been surgically exposed.

### II. SELECTED EMBODIMENTS OF NEURAL RADIANCE FIELD (NERF) TRAINING AND RENDERING USING A MOBILE CAMERA ARRAY

FIG. 4 is a flow diagram of a process or method 430 for generating a three-dimensional (3D) view of the scene 108 from data captured by the sensor array 110 in accordance with embodiments of the present technology. The 3D view can be a photoreal 3D view of the scene 108 from a novel perspective—that is, for example, an interpolated view from a perspective that does not correspond directly to any of the cameras 112. Although some features of the method 430 are described in the context of the system 100 shown in FIGS. 1-3 for the sake of illustration, one skilled in the art will readily understand that the method 430 can be carried out using other suitable systems and/or devices described herein.

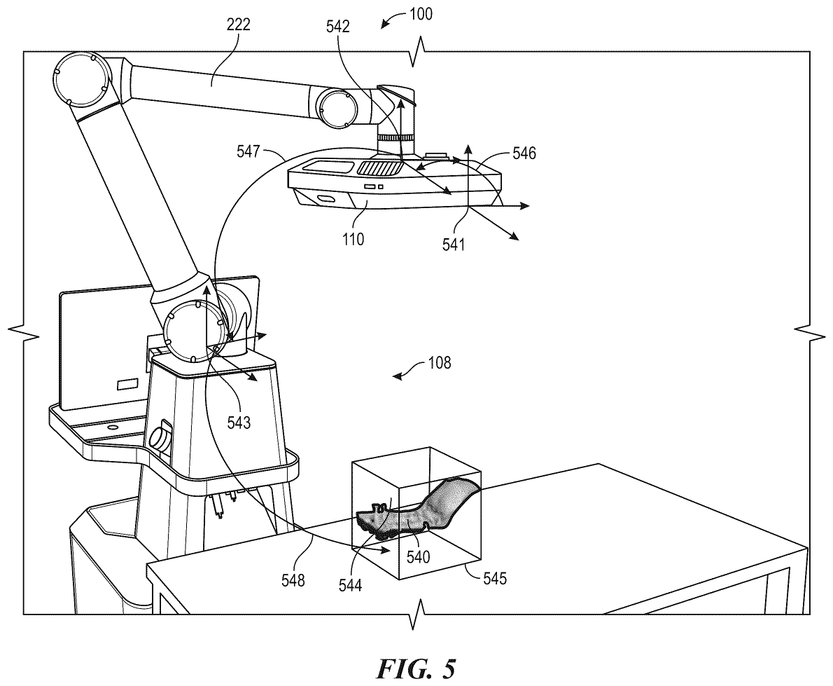

At block 431, the method can include co-calibrating (i) the sensor array 110 including the cameras 112 and the depth sensor 114, (ii) the robotic mover 222, and (iii) a portion (e.g., target volume) of the scene 108. For example, FIG. 5 is a partially-schematic perspective view of the system 100 in accordance with embodiments of the present technology. In the illustrated embodiment, the scene 108 includes a target 540, such as a portion of a spine of a patient undergoing a spinal surgical procedure, positioned within a target volume 545. Each of the cameras 112 and the depth sensor 114 (FIG. 1) have their own sensor reference frame 541 (which can also be referred to as a coordinate frame; only one shown in FIG. 5), the physical structure of the sensor array 110 has an array reference frame 542, the robotic mover 222 has a mover reference frame 543, and the target 540 and/or target volume 545 have a target reference frame 544 (collectively “reference frames 541-544”). The calibration at block 431 determines the spatial and positional relationships between the different reference frames 541-544 such that data captured in one of the reference frames 541-544 can be translated/transformed to another one of the reference frames 541-544 and/or such that their relative positions can be tracked. For example, FIG. 5 illustrates a sensor-array transformation 546 (only one shown in FIG. 5) between sensor reference frames 541 and the array reference frame 542, an array-mover transformation 547 between the array reference frame 542 and the mover reference frame 543, and a mover-target transformation 548 between the mover reference frame 543 and the target reference frame 544 (collectively “transformations 546-548”).

More specifically, referring to FIGS. 1-5 together, block 431 can include initially calibrating (e.g., both intrinsically and extrinsically) the sensor array 110 to, for example, determine a pose (e.g., a position and orientation) for each of the cameras 112 and the depth sensor 114 (and the trackers 113) in three-dimensional (3D) space with respect to a shared origin (e.g., with respect to the array reference frame 542). This can include calibrating (e.g., both intrinsically and extrinsically) the cameras 112 such that, after calibration, image data from each of the spaced apart cameras 112 can be represented in the same reference frame, for example, with a measured transformation (e.g., the sensor-array transformation 546) between the individual sensor reference frames 541 of each of the cameras 112. In some embodiments, the processing device 102 performs a calibration process to detect the positions and orientation of each of the cameras 112 in 3D space with respect to a shared origin and/or an amount of overlap in their respective fields of view. For example, the processing device 102 can (i) process captured images from each of the cameras 112 including fiducial markers placed in the scene 108 and (ii) perform an optimization over the camera parameters and distortion coefficients to minimize reprojection error for key points (e.g., points corresponding to the fiducial markers). In some embodiments, the processing device 102 performs the calibration process by correlating feature points across different cameras views. The correlated features can be, for example, reflective marker centroids from binary images, scale-invariant feature transforms (SIFT) features from grayscale or color images, and/or the like. In some embodiments, the processing device 102 extracts feature points from a target (e.g., a ChArUco target) imaged by the cameras 112 and processes the feature points with the OpenCV camera calibration routine. In other embodiments, such a calibration can be performed with a Halcon circle target or other custom target with well-defined feature points with known locations. In some embodiments, further calibration refinement can be carried out using bundle analysis and/or other suitable techniques. The depth sensor 114 can similarly be calibrated such that, after calibration, depth data from each of the spaced apart depth cameras 118 can be represented in the same reference frame (e.g., with a measured transformation between the individual sensor reference frames 451 of each of the depth cameras 118). The calibration process for the depth cameras 118 can be generally similar or identical to those of the cameras 112 described in detail above. For example, the processing device 102 can extract feature points from a target imaged by the depth cameras 118 and match the feature points across the different views of the depth cameras 118.

Accordingly, the individual sensor reference frames 541 can be mapped via accurate and consistent transformations (e.g., the sensor-array transformation 546) to the array reference frame 542. The array reference frame 542 can be calibrated to the mover 222 based on a known geometry and/or range of movement of the mover 222. That is, the array reference frame 542 can be mapped via an accurate and consistent transformation (e.g., the array-mover transformation 547) to the mover reference frame 543 by the known geometry and movement constraints of the mover 222 relative to the sensor array 110. Such properties can be determined, for example, at the time of manufacturing the system 100.

Lastly, the array reference frame 542 can be calibrated to the target reference frame 544 including the target 540 and/or the target volume 545. For example, one or more ArUco or other markers can be placed within the scene 108 and imaged via the sensor array 110. More specifically, an ArUco reference board or other co-calibration target can be positioned within the scene 108 and imaged with the sensor array 110. The ArUco reference board can have a pattern and/or markers that share a known common origin and coordinate frame (e.g., defined as the target reference frame 544) that allows for co-calibration of the array reference frame 542 to the target reference frame 544.

Accordingly, after calibration, the system 100 stores accurate and consistent transformations 546-548 between the reference frames 541-544 of the individual cameras 112, the depth sensor 114, the mover 222, and the target 540 and/or target volume 545 within the scene 108. That is, the calibration can include determining (e.g., estimating) the pose of each subsystem of the system 100 including the cameras 112, the depth sensor 114, and the mover 222, and then determining a transformation between each subsystem within the target reference frame 544. In some aspects of the present technology, the stored and fixed transformations 546-548 enable the rapid determination of a pose of each the cameras 112 and the depth sensor 114 relative to the target 540 and/or the target volume 545. That is homography can be maintained even when the sensor array 110 is moved relative to the scene 108.

In some embodiments, the various calibration processes at block 431 can include some features generally or similar identical to any of the calibration processes described in (i) “U.S. patent application Ser. No. 18/327,495, filed Jun. 1, 2023, and titled “METHODS AND SYSTEMS FOR CALIBRATING AND/OR VERIFYING A CALIBRATION OF AN IMAGING SYSTEM SUCH AS A SURGICAL IMAGING SYSTEM, (ii) U.S. patent application Ser. No. 18/314,733, filed May 9, 2023, and titled “METHODS AND SYSTEMS FOR CALIBRATING INSTRUMENTS WITHIN AN IMAGING SYSTEM, SUCH AS A SURGICAL IMAGING SYSTEM,” and/or (iii) U.S. patent application Ser. No. 15/930,305, filed May 12, 2020, and titled “METHODS AND SYSTEMS FOR IMAGING A SCENE, SUCH AS A MEDICAL SCENE, AND TRACKING OBJECTS WITHIN THE SCENE,” each of which is incorporated herein by reference in its entirety.

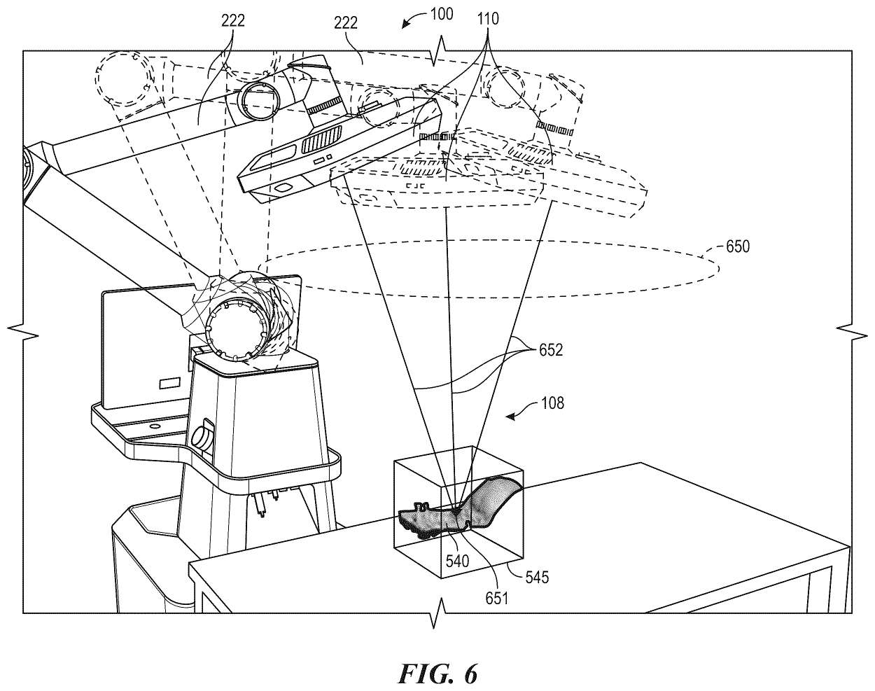

At block 432, the method 430 can include moving the sensor array 110 to and/or through multiple different positions relative to the target 540 and the target volume 545 (FIG. 5) within the scene 108. For example, FIG. 6 is a partially-schematic perspective view of the system 100 showing the sensor array 110 moved via the robotic mover 222 and positioned at multiple different positions relative to the scene 108 including the target 540 and the target volume 545 in accordance with embodiments of the present technology. In some embodiments, the sensor array 110 can be moved in a regular pattern along, for example, a path 650. In some embodiments, the path 650 is circular. In other embodiments, the path 650 can be oval-shaped, otherwise-shaped, symmetric, asymmetric, etc. Notably, the sensor array 110 has a different perspective and pose relative to the target 540 and target volume 545 at each position. In some embodiments, the robotic mover 222 can move the sensor array 110 along the path 650 such that the sensor array 110 maintains the same or substantially same focus point 651 of the target 540 and/or within the target volume 545. More particularly, for example, in the illustrated embodiment the sensor array 110 has an optical axis 652 that is consistently aligned with the focus point 651 during movement of the sensor array 110 along the path 650. Referring to FIGS. 3 and 6, in some embodiments the optical axis 652 can extend through the common point at which the focal axes 329 of the cameras 112 converge.

At block 433, the method 430 can include capturing RGB image data with the cameras 112 and depth data with the depth sensor 114 at each (or at least some) of the positions to which the sensor array 110 is moved at block 432. In some embodiments, the number of positions can comprise a predetermined number of positions (e.g., two, three, four, ten, or more) along the path 650 (FIG. 5). In some embodiments, the RGB image data and the depth data can be collected at a predetermined temporal or spatial interval as the sensor array 110 moves along the path 650. In some embodiments, the RGB image data and the depth data can be collected at many positions along the path 650 in real-time or near real-time such that the number of data collection positions is very large (e.g., including 100s, 1000s, or more positions). The captured RGB image data and depth data can be transferred to the processing device 102 for further processing described below. In some embodiments, the depth data comprises a point cloud and/or a 3D mesh of that represents the surface geometry of the scene 108 including at least a portion of the target 540 within the target volume 545 shown in FIGS. 5 and 6.

At block 434, the method 430 can include determining the poses (e.g., position in space, orientation, and/or the like) of the cameras 112 and the pose of the depth sensor 114 used to capture the RGB image data and depth data, respectively, at block 433. The poses of the cameras 112 and the pose of the depth sensor 114 can be determined based on the calibration transformations 546-548 determined at block 431. In some aspects of the present technology, the calibration transformations 546-548 can be determined/retrieved in real-time or substantially real-time because the system 100 maintains homography even as the mover 222 moves the sensor array 110 including the cameras 112 and the depth sensor 114 about the scene 108. In contrast, conventional imaging systems utilizing neural radiance field (NeRF) rendering algorithms typically require that the pose of cameras be computed via images captured by the cameras. For example, common features can be compared between images captured by different cameras to determine homography. However, such computational methods for determining the poses of the cameras based on captured images are computationally expensive and therefore cannot be performed in real-time or near real-time.

At block 435, the method 430 can include inserting the RGB data captured by the cameras 112 into a radiance volume of a neural radiance field (NeRF) algorithm based on the determined poses of the cameras 112 at the multiple positions. The radiance volume can correspond to the target volume 545 (FIGS. 5 and 6). The RGB data comprises a set of sparse input views of the radiance volume and serves as training data for a neural network. The radiance volume represents a volume probability (e.g., density) function that indicates how much radiance and/or luminance is accumulated by a ray passing through a 3D point (e.g., having x, y, z coordinates). The NeRF algorithm can comprise a fully-connected neural network that can generate novel views of complex 3D scenes (e.g., the scene 108) based on a partial set of 2D images (e.g., RGB images captured by the cameras 112). The NeRF algorithm is trained to use a rendering loss to reproduce input views of the scene 108. It works by taking input images representing the scene 108 and interpolating between them to render one complete scene. Accordingly, the radiance volume can be represented in the NeRF algorithm by a fully-connected neural network. More specifically, because the RGB input data to the NeRF algorithm captured by the cameras 112 is sparse (e.g., comprising a few images), the radiance volume is trained (e.g., optimized) to “fill” the radiance volume (e.g., by interpolation) so that many synthetic views can be generated, as described in greater detail below with reference to blocks 437-439.

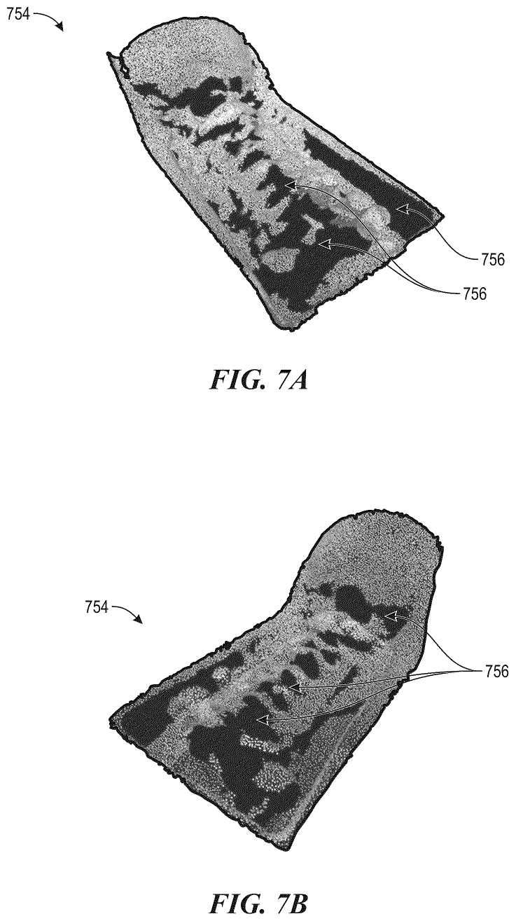

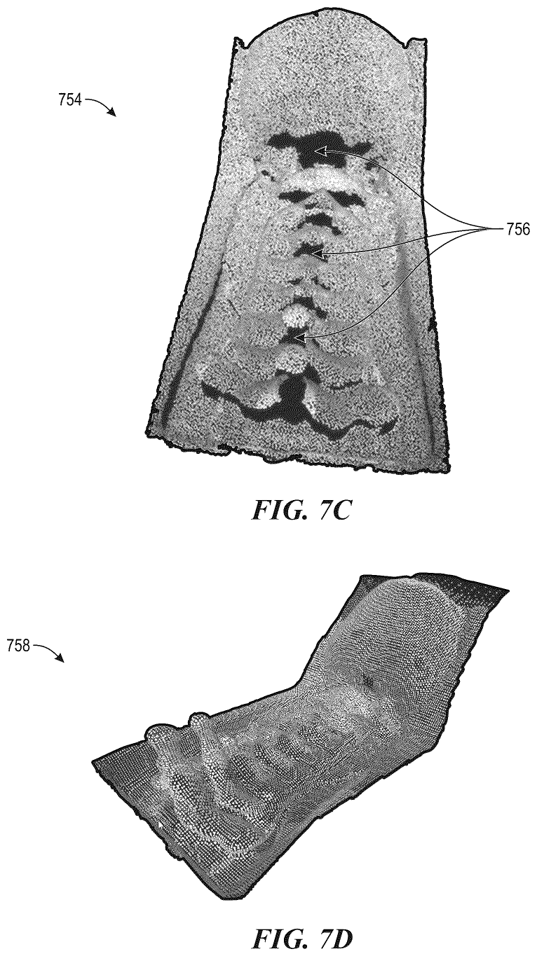

At block 436, the method 430 can include generating a unified depth map based on the depth data captured by the depth sensor 114 and the determined poses of the depth sensor 114 at the multiple positions. That is, the depth data captured from the depth sensor 114 at each of the positions at block 434 can be combined to form a unified depth map that provides a higher fidelity 3D model of the scene 108 than the depth data captured by the depth sensor 114 at a single one of the positions. For example, FIGS. 7A-7C are schematic perspective views of different 3D depth maps 754 of the target 540 (FIGS. 5 and 6) captured by the depth sensor 114 at different positions about the scene 108 in accordance with embodiments of the present technology. Referring to FIGS. 6-7C, the depth maps 754 can be point clouds comprising a plurality of points representing the depth of the scene 108 within the target volume 545 and including the target 540. Depending on the pose of the depth sensor 114 at each position, the resulting depth map 754 may include regions of occlusion 756 with few or no points (e.g., little or no depth data) where the target 540 is at least partially occluded and/or obscured. For example, where the target 540 is the spine of a patient, the spine may include regions of vertebrae that extend generally orthogonal to the cameras 118 of the depth sensor 114 (FIG. 1) at a given position and that are therefore difficult to capture depth data of. Likewise, regions of the spine and/or adjacent tissue may be positioned behind other regions of spine and/or tissue and therefore occluded from the field of view of the depth sensor 114 at a given position.

Accordingly, the depth maps 754 captured at the different positions can be combined to generate a higher-fidelity depth map. FIG. 7D, for example, is a schematic perspective view of a combined or unified depth map 758 that combines the different depth maps 754 shown in FIGS. 7A-7C. Referring to FIGS. 7A-7D, the unified depth map 758 includes no or fewer regions of occlusion 756 than the individual depth maps 754 from which it is combined. In some embodiments, the depth maps 754 can be combined to form/generate the unified depth map 758 based on the determined pose of the depth sensor 114 (block 434) at each of the positions from which the individual depth maps 754 are captured. In some aspects of the present technology, the unified depth map 758 can be generated in real-time or substantially real-time because the system 100 maintains homography even as the mover 222 moves the sensor array 110 including the depth sensor 114 about the scene 108. In some embodiments, the processing device 102 can generate a 3D mesh from the unified depth map 758 representative of the depth of the scene 108.

In some aspects of the present technology, using the dedicated depth sensor 114 to generate the depth data and subsequent unified depth map can increase processing times and depth accuracy as compared to reconstructing/generating a depth map from the image data from the cameras 112. For example, using the image data from the cameras 112 to reconstruct the depth of the scene 108 can require processing of the image data to extract feature points and then identifying which feature points are correspondences between images. Such a process can be noisy, and can also result in a sparse depth map that requires additional processing to be smoothed or filled in. In contrast, for example, the projection of a continuous pattern/texture over the scene 108 via the projector 116 allows for robust block matching to produce a highly complete depth map at each position of the sensor array 110. Moreover, in some embodiments the depth cameras 118 can be stereo cameras with epipolar constraints that accelerate depth processing compared to the collection of cameras 112 (e.g., RGB cameras).

At block 437, the method 430 can include applying depth supervision within the NeRF algorithm based on the generated unified depth map. For example, the NeRF algorithm can receive the unified depth map as an input and use the unified depth map (e.g., a point cloud or subsequently generated mesh) as a boundary condition (e.g., region of constraint) to, for example, restrain training to regions near the surface of the target 540 (FIGS. 5 and 6) within the scene 108. For example, the NeRF algorithm can filter the radiance volume based on the unified depth map. For example, the NeRF algorithm can apply the unified depth map as a first-order approximation of the surface of the target 540 within the scene 108—and thus as an approximation of the radiance field used in the training of the NeRF algorithm. That is, the unified depth map represents a contour in the 3D volume of the scene 108 to be rendered by the NeRF algorithm that can be propagated through the algorithm to accelerate processing (e.g., as compared to a brute-force computational approach) by omitting training and processing of regions of the radiance volume for which there is not RGB image data.

At block 438, the method 430 can include updating the training of the radiance volume based on the captured RGB data and the depth supervision. For example, various weights of the NeRF algorithm can be refined/optimized based on the training. More specifically, the radiance volume can be trained (e.g., optimized) to “fill” the radiance volume (e.g., by interpolation) with data based on the sparse input views from the cameras 112 so that many synthetic views can be generated.

At block 439, the method 430 can include rendering an output image of the scene 108 including the target 540 within the target volume 545 (FIGS. 5 and 6) based on a specified observer pose using the updated NeRF algorithm. The observer pose can be input via the input controller 106 based on user driven changes, as described in detail above. The output image can be a novel/synthetic 3D virtual view of the scene 108. Specifically, the trained NeRF algorithm can take as an input the specified observer pose (e.g., a camera pose) and output the rendered output image of the target volume 545. In some embodiments, the rendering can occur in real-time or near real-time and the output image can be output to, for example, the display device 104 and/or the secondary display 226 (FIGS. 1 and 2) for display to a user of the system 100. In some embodiments, multiple output images can be rendered at block 439. For example, the trained NeRF algorithm can render an image from multiple different perspectives.

At block 440, the method 430 can include removing (e.g., deleting) the training data inserted as training data into the radiance volume at block 435. The method 430 can then return to block 432 and proceed again. That is, the method 430 can loop through blocks 432-440 in a continuous manner to (i) capture new RGB image data from the cameras 112 and new depth data from the depth sensor 114, (ii) utilize the captured RGB image data and depth data to update the training of the NeRF algorithm, and (iii) render an updated 3D output image of the scene 108 at a desired viewer perspective. For example, referring to FIG. 6, the mover 222 can repeatedly move the sensor array 110 along the path 650 and/or another path such that the sensor array 110 loiters above the scene 108. After each traverse of the path 650—or after traverse of some subset of the path 650 including multiple positions—the method 430 can proceed through blocks 432-440 to update the training and the subsequently rendered output image of the scene 108. As described in detail above, in some aspects of the present technology blocks 432-440 can be performed in real-time or near real-time because the system maintains homography throughout and/or because the system 100 utilizes depth supervision to optimize processing to occur only near the actual surfaces of the target 540. Accordingly, the method 430 can continually capture and feed new training data into the NeRF algorithm, before subsequently rendering an updated output image of the scene 108 in a manner that scales to produce a real-time or near real-time NeRF update of the scene 108—even where the scene 108 is dynamic (e.g., including changes). For example, the method 430 can continually proceed through blocks 432-440 at a frequency of 5-10 Hertz or greater to improve the training and to capture dynamic changes to the scene 108, such as changes to the target 540 resulting from manipulation of the target 540 during a surgical procedure (e.g., a spinal surgical procedure).

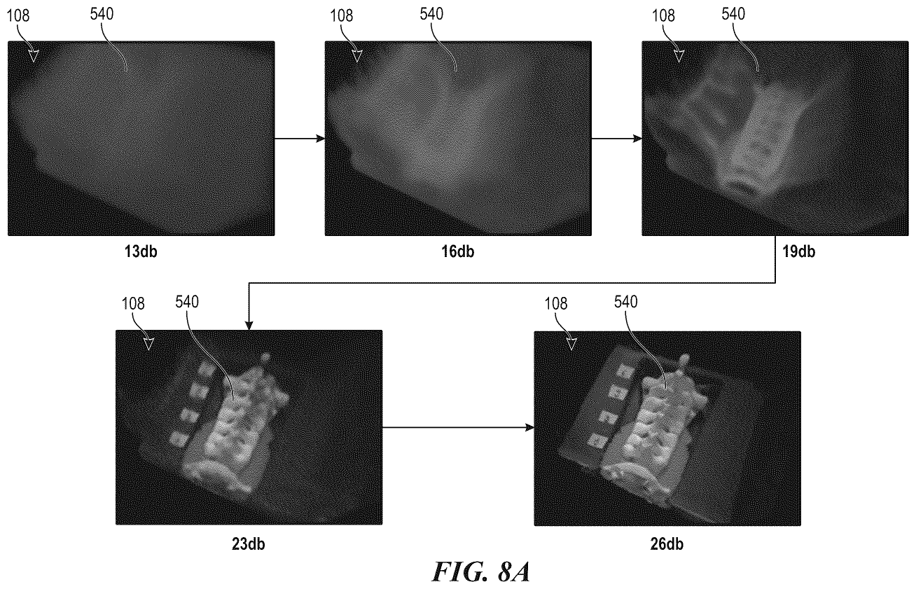

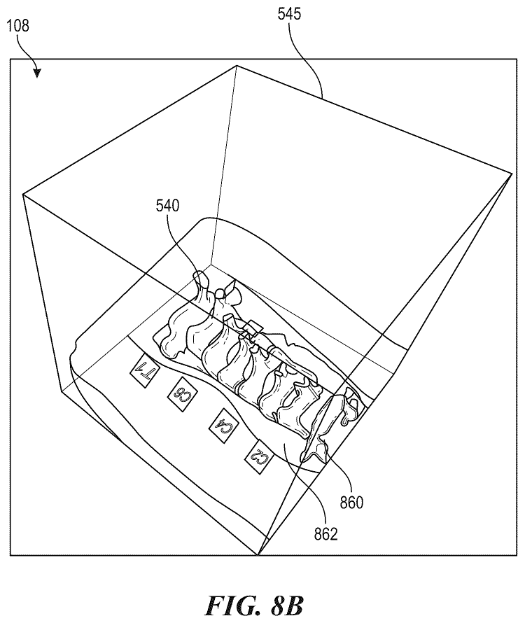

FIG. 8A illustrates several rendered views of the scene 108 including the target 540 in accordance with embodiments of the present technology. As indicated by arrows, the rendered view improves as the method 430 loops through blocks 432-440 in real-time or near-real time to update the training of the NeRF algorithm. Specifically, a decibel level of a signal-to-noise ratio (e.g., a peak signal-to-noise ratio (PSNR)) can increase as indicated by the decibel levels shown in FIG. 8A during iterative updating of the NeRF algorithm as a “radiance fog” of the rendered output images decreases. FIG. 8B illustrates a finally rendered output image of the scene 108 including the target 540 within the target volume 545 in accordance with embodiments of the present technology. In the illustrated embodiment, the target 540 can have a photoreal aspect. In some embodiments, the output image includes accurate color information of the target 540 that can facilitate differentiation between, for example, bone 860 (e.g., vertebral bone), tissue 862, and/or other anatomical components.

Referring to FIG. 4, while many aspects of the method 430 are described in the context of a NeRF algorithm, rendering algorithms other than NeRF algorithms can be used in the method 430. For example, in some embodiments the method 430 employs a Gaussian splatting algorithm (e.g., a 3D Gaussian splatting algorithm) alternatively or additionally to a NeRF algorithm. Gaussian splatting is a rendering technique that uses machine learning to represent 3D scenes as a set of translucent Gaussian spheres. During training, Gaussian splatting algorithms typically use a machine learning method such as steepest descent (e.g., stochastic gradient descent) to calculate the parameters of each Gaussian. While Gaussian splatting algorithms are similar to NeRF algorithms, and can be utilized in the method 430 in the same or generally similar manner, Gaussian splatting algorithms differ from NeRF algorithms in some aspects. For example, whereas NeRF algorithms carry out ray marching through a light field volume for rendering, Gaussian splatting algorithms employ rasterization (e.g., tile-based rasterization) for rendering. The rasterization approach of Gaussian splatting algorithms can provide for rendering speeds that are substantially greater (e.g., about 50 times greater) than comparable NeRF algorithms. However, Gaussian splatting algorithms typically require much more storage space than NeRF algorithms. Accordingly, the method 430 can include utilizing a NeRF algorithm, a Gaussian splatting algorithm, and/or other like algorithms depending on the particular application and system limitations.

### III. SELECT EMBODIMENTS OF DISPLAYING NERF AND/OR GAUSSIAN SPLATTING RENDERED IMAGES

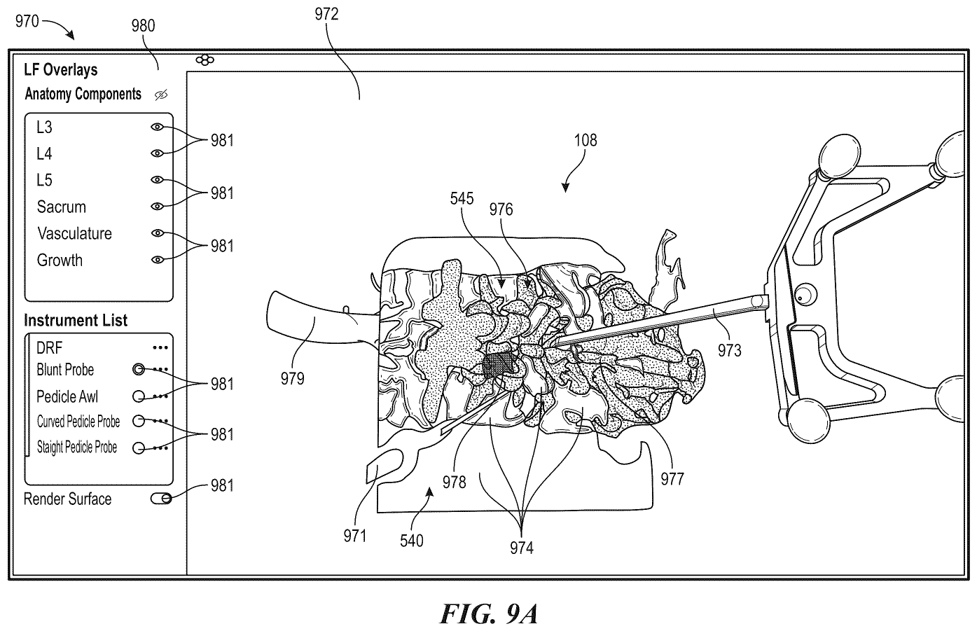

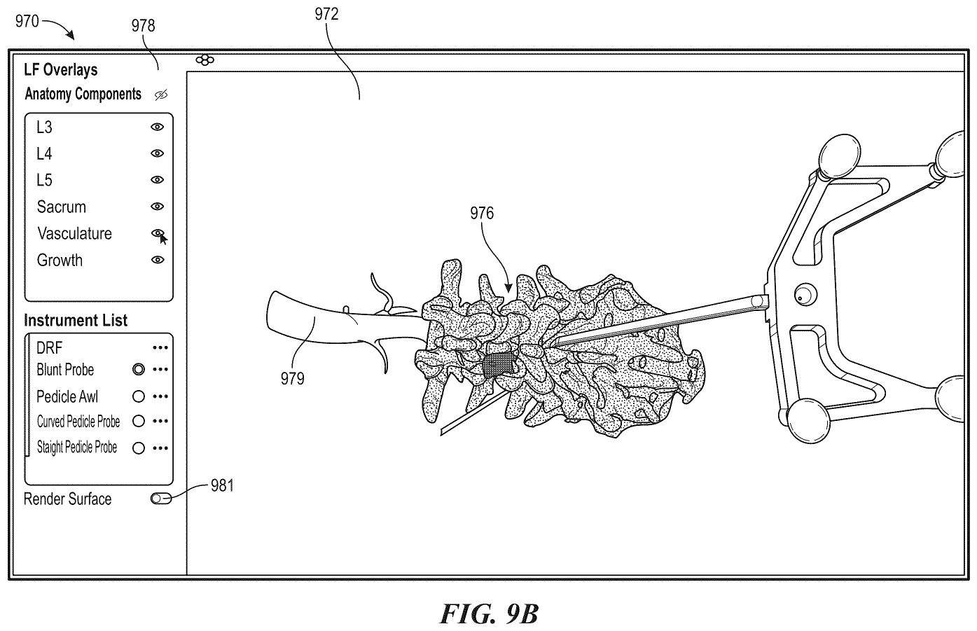

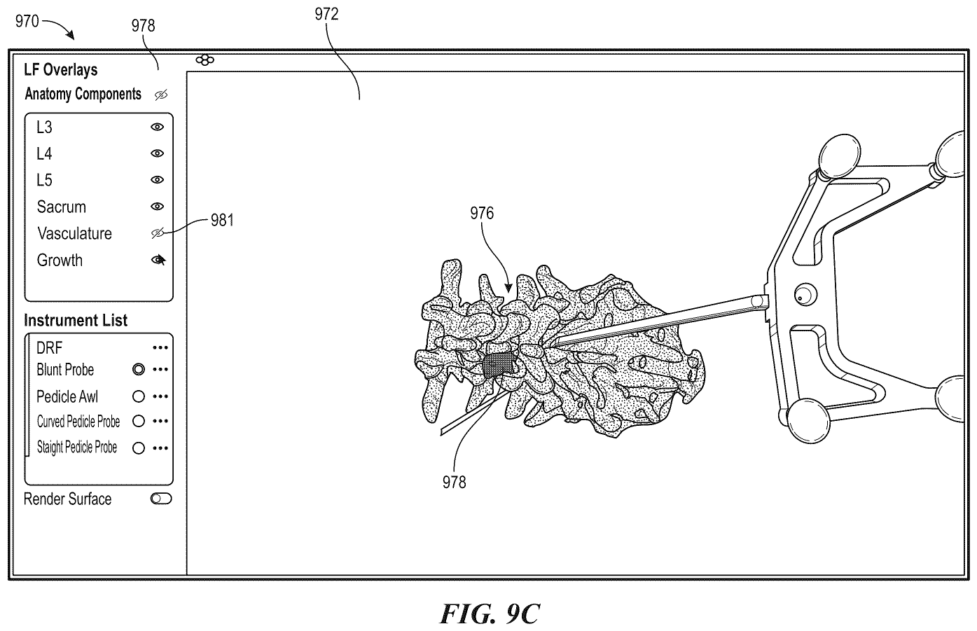

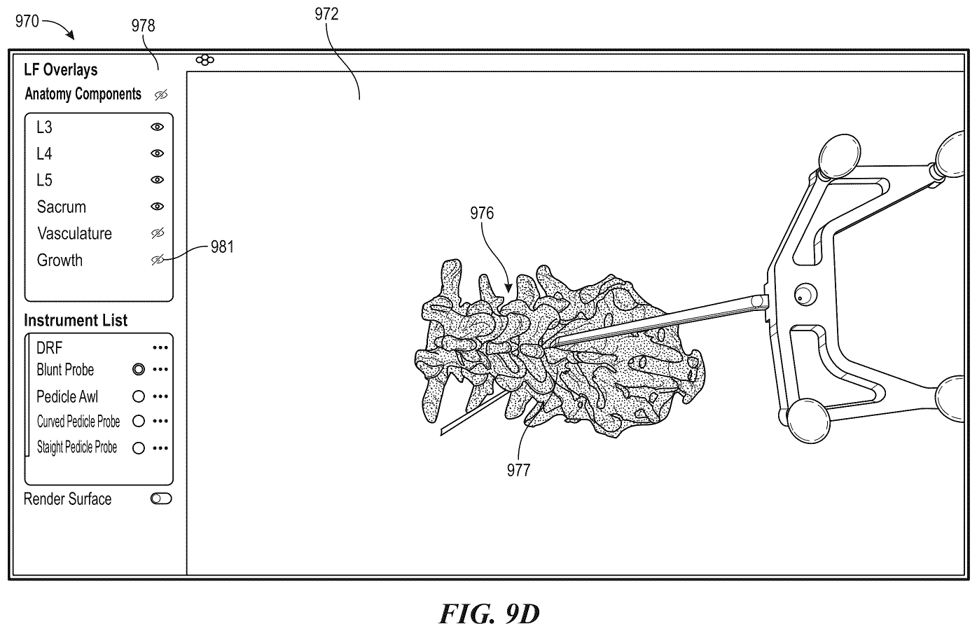

In some embodiments, a NeRF-rendered output image and/or a Gaussian splatting-rendered output image generated by the method 430 of FIG. 4 can be registered to and combined with one or more other imaging modalities to produce a combined or fused output image. FIGS. 9A-9D, for example, are different views of a user interface 970 (e.g., a display) visible to a user of the system 100 via the display device 104 (e.g., a head-mounted display device), the secondary display 226, and/or another display of the system 100 in accordance with embodiments of the present technology. The user interface 970 can include some features that are at least generally similar in structure and/or function, or identical in structure and/or function, to any of the user interfaces described in U.S. patent application Ser. No. 17/864,065, filed Jul. 12, 2022, and titled “METHODS AND SYSTEMS FOR DISPLAYING PREOPERATIVE AND INTRAOPERATIVE IMAGE DATA OF A SCENE,” which is incorporated herein by reference in its entirety.

Referring to FIG. 9A, the user interface 970 includes a viewport or display panel 972 configured to display both (i) a 3D output image 974 of the target 540 (e.g., a spine and/or surrounding anatomy) within the target volume 545 and (ii) initial 3D image data 976 of the target 540. The display panel 972 can further display (e.g., overlay) a representation of one or more instruments (e.g., tools, objects), such as surgical instruments, and/or the 3D output image 974 can include image data of such instruments. For example, in the illustrated embodiment an instrument 971 (e.g., a surgical probe) and a dynamic reference frame (DRF) marker 973 are displayed on the display panel 972.

The 3D output image 974 can be generated using the method 430 described in detail above with reference to FIGS. 4-8B and can, for example, correspond to the output image shown in FIG. 8B. In some embodiments, the initial 3D image data 976 (e.g., previously-captured image data) is preoperative image data comprising 3D geometric and/or volumetric data, such as computed tomography (CT) scan data, magnetic resonance imaging (MRI) scan data, ultrasound image data, fluoroscopic image data, and/or other medical or other image data. More specifically, the 3D image data 976 can comprise segmented MRI scan data, merged and segmented MRI and CT scan data, and/or the like. For example, in the illustrated embodiment the initial 3D image data 976 includes segmented 3D geometric and/or volumetric data of a spine 977 of a patient, a growth 978 (e.g., tumor) on the spine 977, and a vasculature 979 proximate to the spine 977. In some embodiments, the initial 3D image data 976 can be captured intraoperatively. For example, the initial 3D image data 976 can comprise 2D or 3D X-ray images, fluoroscopic images, CT images, MRI images, etc., and combinations thereof, captured of the patient within an operating room. In some embodiments, the initial 3D image data 976 comprises a point cloud, three-dimensional (3D) mesh, and/or another 3D data set.

The initial 3D image data 976 can be registered to the physical scene 108 and the 3D output image 974 using a suitable registration process. For example, the initial 3D image data 976 can be registered to the physical scene 108 by comparing corresponding points in both the 3D image data 976 and the physical scene 108. For example, a user can touch the instrument 971 to points in the physical scene 108 corresponding to identified points in the initial 3D image data 976, such as pre-planned screw entry points on the patient's spine 977. The system 100 can then generate a registration transformation between the initial 3D image data 976 and the physical scene 108 by comparing the points. The registration can be updated continuously by tracking the DRF marker 973 (e.g., with the trackers 113 of FIG. 1) and/or using another suitable process (e.g., a markerless registration process), such as of the type described in U.S. patent application Ser. No. 18/084,389, filed Dec. 19, 2022, and titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” which is incorporated herein by reference in its entirety.

In the illustrated embodiment, the display panel 972 provides a fused view/overlay of the 3D output image 974 and the initial 3D image data 976 that allows a user (e.g., a physician, a surgeon) to visualize the target 540 and/or surrounding anatomy as well as the (e.g., previously-captured) segmented images of the target 540. Such a fused view can provide sufficient information to the user to allow them to navigate the instrument 971 and/or other instruments relative to the target 540 during a procedure, such as a spinal surgical procedure.

In the illustrated embodiment, the user interface 970 further includes a selection panel 980 including a plurality of icons 981 that are selectable (e.g., toggleable) by a user via a user input device (e.g., a keyboard, a mouse, verbal command device, etc.) to display more or less visual information on the display panel 972. For example, FIG. 9B illustrates the user interface 970 after deselection of a “Render Surface” icon 981 in the selection panel 980. After deselection of the “Render Surface” icon 981, the display panel 972 no longer displays the 3D output image 974 (FIG. 9A)—leaving, for example, the initial 3D image data 976 on display. The “Render Surface” icon 981 can be toggled multiple times to selectively display or not display the 3D output image 974. Similarly, FIG. 9C illustrates the user interface 970 after deselection of a “Vasculature” icon 981 in the selection panel 980. After deselection of the “Vasculature” icon 981, the display panel 972 no longer displays the segmented vasculature portion 979 (FIGS. 9A and 9B) of the initial 3D image data 976. The “Vasculature” icon 981 can be toggled multiple times to selectively display or not display the vasculature portion 979 of the initial 3D image data 976. Likewise, FIG. 9D illustrates the user interface 970 after deselection of a “Growth” icon 981 in the selection panel 980. After deselection of the “Growth” icon 981, the display panel 972 no longer displays the segmented growth portion 978 (FIGS. 9A and 9C) of the initial 3D image data 976. The “Growth” icon 981 can be toggled multiple times to selectively display or not display the growth portion 978 of the initial 3D image data 976. Other ones of the icons 981 can be selectively toggled to display different visual representations and/or portions of the image data.

### IV. ADDITIONAL EXAMPLES

The following examples are illustrative of several embodiments of the present technology:

1. A method of generating a three-dimensional (3D) image of a target volume within a scene, the method comprising:
moving a sensor array through multiple different positions about the scene relative to the target volume;
at each of the positions, capturing (a) RGB image data of the target volume with multiple RGB cameras of the sensor array and (b) depth data of the target volume with a depth sensor of the sensor array;
determining poses of the RBG cameras and a pose of the depth sensor at each of the positions;
inserting the RGB image data as training data into a radiance volume of a neural radiance field (NeRF) algorithm based on the determined poses of the RGB cameras at each of the positions;
at least partially combining the depth data from the depth sensor to generate a unified depth map based on the determined pose of the depth sensor at each of the positions;
training the radiance volume based on the RGB image data while constraining the training based on the unified depth map; and
rendering the 3D image of the target volume based on a specified observer pose using the NeRF algorithm.

2. The method of example 1 wherein the RBG cameras and the depth sensor are fixed to a frame of the sensor array.

3. The method of example 1 or example 2 wherein the sensor array has an optical axis, and wherein moving the sensor array about the scene comprises moving the sensor array such that the optical axis is continually aligned with a focus point within the target volume.

4. The method of any one of examples 1-3 wherein moving the sensor array about the scene comprises moving the sensor array via a robotically-controlled arm.

5. The method of example 4 wherein the method further comprises determining registration transformations between reference frames of the RBG cameras, a reference frame of the depth sensor, a reference frame of the sensor array, a reference frame of the robotically-controlled arm, and a reference frame of the target volume.

6. The method of example 5 wherein determining the poses of the RBG cameras and the poses of the depth sensor is based on the registration transforms.

7. The method of any one of examples 1-6 wherein the scene is a surgical scene, and wherein the target volume includes a surgically-exposed portion of a patient.

8. A method of generating a three-dimensional (3D) image of a target volume within a scene, the method comprising:
co-calibrating (a) a sensor array including a plurality of RGB cameras and a depth sensor, (b) a robotic mover coupled to the sensor array, and (c) the target volume;
moving the sensor array through multiple different positions about the scene relative to the target volume;
at each of the positions, capturing (a) RGB image data of the target volume with the RGB cameras and (b) depth data of the target volume with the depth sensor;
determining poses of the RBG cameras and a pose of the depth sensor at each of the positions based on the co-calibration; and
utilizing the RGB image data, the depth data, and the determined poses of the RGB cameras and the pose of the depth sensor at each of the positions in a neural radiance field (NeRF) algorithm and/or a Gaussian splatting algorithm to render the 3D image of the target volume based on a specified observer pose.

9. The method of example 8 wherein utilizing the RGB image data, the depth data, and the determined poses of the RGB cameras and the pose of the depth sensor at each of the positions in a neural radiance field (NeRF) algorithm and/or a Gaussian splatting algorithm comprises—
inserting the RGB image data as training data into a radiance volume of a neural radiance field (NeRF) algorithm based on the determined poses of the RGB cameras at each of the positions;
at least partially combining the depth data from the depth sensor to generate a unified depth map based on the determined pose of the depth sensor at each of the positions;
training the radiance volume based on the captured RGB data while constraining the training based on the unified depth map; and
rendering the 3D image of the target volume based on the specified observer pose using the NeRF algorithm.

10. The method of example 8 wherein utilizing the RGB image data, the depth data, and the determined poses of the RGB cameras and the pose of the depth sensor at each of the positions in a neural radiance field (NeRF) algorithm and/or a Gaussian splatting algorithm comprises utilizing the image data and the depth data in the Gaussian splatting algorithm.

11. The method of example 8 or example 9 wherein utilizing the RGB image data, the depth data, and the determined poses of the RGB cameras and the pose of the depth sensor at each of the positions in a neural radiance field (NeRF) algorithm and/or a Gaussian splatting algorithm comprises utilizing the image data and the depth data in the NeRF algorithm.

12. The method of any one of examples 8-11 wherein moving the sensor array about the scene comprises moving the sensor array via a robotically-controlled arm.

13. The method of any one of examples 8-12 wherein the RBG cameras and the depth sensor are fixed to a frame of the sensor array.

14. The method of any one of examples 8-13 wherein the sensor array has an optical axis, and wherein moving the sensor array about the scene comprises moving the sensor array such that the optical axis is continually aligned with a focus point within the target volume.

15. A system for generating a three-dimensional (3D) image of a target volume within a scene, comprising:
a sensor array including multiple RGB cameras and a depth sensor, wherein the RGB cameras are configured to capture RGB image data of the target volume, and wherein the depth sensor is configured to capture depth data of the target volume;
a movable arm coupled to the sensor array and configured to move the sensor array through multiple different positions about the scene relative to the target volume; and
a processing device programmed with non-transitory computer readable instructions that, when executed by the processing device, cause the processing device to—
   receive RGB image data of the target volume captured by the RGB cameras at the multiple different positions;
   receive depth data of the target volume captured by the depth sensor at the multiple different positions;
   determine poses of the RBG cameras and a pose of the depth sensor at each of the positions;
   insert the RGB image data as training data into a radiance volume of a neural radiance field (NeRF) algorithm based on the determined poses of the RGB cameras at each of the positions;
   at least partially combine the depth data from the depth sensor to generate a unified depth map based on the determined pose of the depth sensor at each of the positions;
   train the radiance volume based on the RGB image data while constraining the training based on the unified depth map; and
   render the 3D image of the target volume based on a specified observer pose using the NeRF algorithm.

16. The system of example 15 wherein the RBG cameras and the depth sensor are fixed to a frame of the sensor array.

17. The system of example 15 or example 16 wherein the sensor array has an optical axis, and wherein the scene movable arm is configured to move the sensor array through the multiple different positions while continually maintaining the optical in alignment with a focus point within the target volume.

18. The system of any one of examples 15-17 wherein the movable arm is configured to be robotically controlled.

19. The system of any one of examples 15-18 wherein the non-transitory computer readable instructions, when executed by the processing device, cause the processing device to determine the poses of the of the RGB cameras and the pose of the depth sensor at each of the positions based on predetermined registration transformations between reference frames of the RGB cameras, a reference frame of the depth sensor, a reference frame of the sensor array, a reference frame of the movable arm, and a reference frame of the target volume.

20. The system of any one of examples 15-19, further comprising a display separate from the sensor array, wherein the non-transitory computer readable instructions, when executed by the processing device, cause the processing device to render the 3D image of the target volume in real time or near real time for display on the display.

21. A method of generating a three-dimensional (3D) image of a target volume within a scene, the method comprising:
moving a sensor array through multiple different positions about the scene relative to the target volume;
at individual ones of the positions, capturing (a) RGB image data of the target volume with multiple RGB cameras of the sensor array and (b) depth data of the target volume with a depth sensor of the sensor array;
determining poses of the RBG cameras and poses of the depth sensor at the individual ones of the positions;
inserting the captured RGB image data as training data into a radiance volume of a neural radiance field (NeRF) algorithm based on the determined poses of the RGB cameras;
at least partially combining the depth data from the depth sensor to generate a unified depth map based on the determined poses of the depth sensor;
training the radiance volume based on the captured RGB data while constraining the training based on the unified depth map; and
rendering the 3D image of the target volume based on a specified observer pose using the NeRF algorithm.

22. The method of example 21 wherein the cameras and the depth sensor are fixed to a frame of the sensor array.

23. The method of example 21 or example 22 wherein the sensor array has an optical axis, and wherein moving the sensor array about the scene comprises moving the sensor array such that the optical axis is continually aligned with a focus point within the target volume.

24. The method of any one of examples 21-23 wherein moving the sensor array about the scene comprises moving the sensor array via a robotically-controlled arm.

25. The method of example 4 wherein the method includes determining registration transformations between reference frames of the cameras, a reference frame of the depth sensor, a reference frame of the sensor array, a reference frame of the robotically-controlled arm, and a reference frame of the target volume.

26. The method of example 5 wherein determining the poses of the RBG cameras and the poses of the depth sensor is based on the registration transforms.

27. A system for generating a three-dimensional (3D) image of a target volume within a scene, comprising:
a sensor array including multiple RGB cameras and a depth sensor, wherein the RGB cameras are configured to capture RGB image data of the target volume, and wherein the depth sensor is configured to capture depth data of the target volume;
a mover coupled to the sensor array and configured to move the sensor array through multiple different positions about the scene relative to the target volume; and
a processing device programmed with non-transitory computer readable instructions that, when executed by the processing device, cause the processing device to carry out of any one of the methods of examples 1-14 and 21-26.

### V. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of generating a three-dimensional (3D) image of a target volume within a scene, the method comprising:
moving a sensor array through multiple different positions about the scene relative to the target volume;
at each of the positions, capturing (a) RGB image data of the target volume with multiple RGB cameras of the sensor array and (b) depth data of the target volume with a depth sensor of the sensor array;
determining poses of the RBG cameras and a pose of the depth sensor at each of the positions;
inserting the RGB image data as training data into a radiance volume of a neural radiance field (NeRF) algorithm based on the determined poses of the RGB cameras at each of the positions;
at least partially combining the depth data from the depth sensor to generate a unified depth map based on the determined pose of the depth sensor at each of the positions;
training the radiance volume based on the RGB image data while constraining the training based on the unified depth map; and
rendering the 3D image of the target volume based on a specified observer pose using the NeRF algorithm.

2. The method of claim 1 wherein the RBG cameras and the depth sensor are fixed to a frame of the sensor array.

3. The method of claim 1 wherein the sensor array has an optical axis, and wherein moving the sensor array about the scene comprises moving the sensor array such that the optical axis is continually aligned with a focus point within the target volume.

4. The method of claim 1 wherein moving the sensor array about the scene comprises moving the sensor array via a robotically-controlled arm.

5. The method of claim 4 wherein the method further comprises determining registration transformations between reference frames of the RBG cameras, a reference frame of the depth sensor, a reference frame of the sensor array, a reference frame of the robotically-controlled arm, and a reference frame of the target volume.

6. The method of claim 5 wherein determining the poses of the RBG cameras and the poses of the depth sensor is based on the registration transforms.

7. The method of claim 1 wherein the scene is a surgical scene, and wherein the target volume includes a surgically-exposed portion of a patient.

8. A method of generating a three-dimensional (3D) image of a target volume within a scene, the method comprising:
co-calibrating (a) a sensor array including a plurality of RGB cameras and a depth sensor, (b) a robotic mover coupled to the sensor array, and (c) the target volume;
moving the sensor array through multiple different positions about the scene relative to the target volume;
at each of the positions, capturing (a) RGB image data of the target volume with the RGB cameras and (b) depth data of the target volume with the depth sensor;
determining poses of the RBG cameras and a pose of the depth sensor at each of the positions based on the co-calibration; and
utilizing the RGB image data, the depth data, and the determined poses of the RGB cameras and the pose of the depth sensor at each of the positions in a neural radiance field (NeRF) algorithm and/or a Gaussian splatting algorithm to render the 3D image of the target volume based on a specified observer pose.

9. The method of claim 8 wherein utilizing the RGB image data, the depth data, and the determined poses of the RGB cameras and the pose of the depth sensor at each of the positions in a neural radiance field (NeRF) algorithm and/or a Gaussian splatting algorithm comprises—
inserting the RGB image data as training data into a radiance volume of a neural radiance field (NeRF) algorithm based on the determined poses of the RGB cameras at each of the positions;
at least partially combining the depth data from the depth sensor to generate a unified depth map based on the determined pose of the depth sensor at each of the positions;
training the radiance volume based on the captured RGB data while constraining the training based on the unified depth map; and
rendering the 3D image of the target volume based on the specified observer pose using the NeRF algorithm.

10. The method of claim 8 wherein utilizing the RGB image data, the depth data, and the determined poses of the RGB cameras and the pose of the depth sensor at each of the positions in a neural radiance field (NeRF) algorithm and/or a Gaussian splatting algorithm comprises utilizing the image data and the depth data in the Gaussian splatting algorithm.

11. The method of claim 8 wherein utilizing the RGB image data, the depth data, and the determined poses of the RGB cameras and the pose of the depth sensor at each of the positions in a neural radiance field (NeRF) algorithm and/or a Gaussian splatting algorithm comprises utilizing the image data and the depth data in the NeRF algorithm.

12. The method of claim 8 wherein moving the sensor array about the scene comprises moving the sensor array via a robotically-controlled arm.

13. The method of claim 8 wherein the RBG cameras and the depth sensor are fixed to a frame of the sensor array.

14. The method of claim 8 wherein the sensor array has an optical axis, and wherein moving the sensor array about the scene comprises moving the sensor array such that the optical axis is continually aligned with a focus point within the target volume.

15. A system for generating a three-dimensional (3D) image of a target volume within a scene, comprising:
a sensor array including multiple RGB cameras and a depth sensor, wherein the RGB cameras are configured to capture RGB image data of the target volume, and wherein the depth sensor is configured to capture depth data of the target volume;
a movable arm coupled to the sensor array and configured to move the sensor array through multiple different positions about the scene relative to the target volume; and
a processing device programmed with non-transitory computer readable instructions that, when executed by the processing device, cause the processing device to—
receive RGB image data of the target volume captured by the RGB cameras at the multiple different positions;
receive depth data of the target volume captured by the depth sensor at the multiple different positions;
determine poses of the RBG cameras and a pose of the depth sensor at each of the positions;
insert the RGB image data as training data into a radiance volume of a neural radiance field (NeRF) algorithm based on the determined poses of the RGB cameras at each of the positions;
at least partially combine the depth data from the depth sensor to generate a unified depth map based on the determined pose of the depth sensor at each of the positions;
train the radiance volume based on the RGB image data while constraining the training based on the unified depth map; and
render the 3D image of the target volume based on a specified observer pose using the NeRF algorithm.

16. The system of claim 15 wherein the RBG cameras and the depth sensor are fixed to a frame of the sensor array.

17. The system of claim 15 wherein the sensor array has an optical axis, and wherein the scene movable arm is configured to move the sensor array through the multiple different positions while continually maintaining the optical in alignment with a focus point within the target volume.

18. The system of claim 15 wherein the movable arm is configured to be robotically controlled.

19. The system of claim 15 wherein the non-transitory computer readable instructions, when executed by the processing device, cause the processing device to determine the poses of the of the RGB cameras and the pose of the depth sensor at each of the positions based on predetermined registration transformations between reference frames of the RGB cameras, a reference frame of the depth sensor, a reference frame of the sensor array, a reference frame of the movable arm, and a reference frame of the target volume.

20. The system of claim 15, further comprising a display separate from the sensor array, wherein the non-transitory computer readable instructions, when executed by the processing device, cause the processing device to render the 3D image of the target volume in real time or near real time for display on the display.
